(19) United States
(12) Patent Application Publication
(10) Pub. No.: US 2026/0364129 A1
(43) Pub. Date: Nov. 19, 2026

(54) BIOFILM-BASED MATERIALS AND METHODS FOR REMOVAL OF MICROPLASTICS AND OTHER SUBSTANCES FROM FLUIDS, THE ENVIRONMENT, AND BIOLOGICAL SYSTEMS

(71) Applicant: QUORUM INNOVATIONS, INC., SARASOTA, FL (US)

(72) Inventors: PHINEAS NICHOLAS MONSUL, SARASOTA, FL (US); NICHOLAS T. MONSUL, SARASOTA, FL (US); EVA A. BERKES, SARASOTA, FL (US); JULIAN NICHOLAS MONSUL, SARASOTA, FL (US); ODED ORON, SARASOTA, FL (US)

(21) Appl. No.: 19/687,468

(22) Filed: May 26, 2026

Related U.S. Application Data
(63) Continuation of application No. PCT/US2026/028789, filed on May 19, 2026.
(60) Provisional application No. 63/808,281, filed on May 19, 2025.
(60) Provisional application No. 63/931,105, filed on Dec. 4, 2025.
(60) Provisional application No. 64/008,307, filed on Mar. 17, 2026.

(51) Int. Cl. A61K 35/747; A61K 9/00

(52) U.S. Cl. CPC A61K 35/747; A61K 9/006

(57) ABSTRACT

The subject invention provides compositions and methods for removing microplastics from fluids, biological systems and other environments. In preferred embodiments, the compositions of the subject invention comprise bacterial biofilm that strongly adheres to microplastics to form an aggregate thereby facilitating the detection of microplastics and/or the removal of microplastics. Microplastics may be removed from, for example, the environment, a fluid or a biological system.

### CROSS-REFERENCE TO RELATED APPLICATIONS

This application is a continuation of International Application No. PCT/US2026/028789, filed May 19, 2026; which claims priority to U.S. Provisional Patent Application Ser. No. 63/808,281, filed May 19, 2025; Ser. No. 63/931,105, filed Dec. 4, 2025; and Ser. No. 64/008,307, filed Mar. 17, 2026, each of which are incorporated herein by reference in their entireties.

### BACKGROUND OF THE INVENTION

The rapid expansion of plastic production since the mid-20th century has brought convenience to human life, from packaging and medical devices to textiles and technology. However, this plastic boom has also introduced a pervasive environmental threat that is only now beginning to be fully understood: microplastics. Defined as plastic particles smaller than five millimeters in diameter, microplastics have infiltrated ecosystems across the planet, reaching even the most remote and seemingly untouched regions.

The problems associated with microplastics are profound and multifaceted, including environmental degradation and human health concerns. As these tiny pollutants accumulate in marine, freshwater, terrestrial, and atmospheric environments, their presence disrupts natural systems and poses increasingly urgent risks to societies.

Microplastics stem from two principal sources: primary microplastics, which are manufactured at small sizes for specific purposes, and secondary microplastics, which result from the fragmentation of larger plastic products.

Primary microplastics are deliberately produced in a microscopic form for use in a range of industrial and commercial applications. One of the most well-known examples is microbeads, which have been used in personal care products such as exfoliating cleansers, toothpaste, and shower gels. These tiny beads, often made of polyethylene or polypropylene, serve as abrasive agents but are easily washed down household drains, eventually reaching rivers, lakes, and oceans.

Beyond personal care, primary microplastics are used in industrial processes. Plastic pellets, known as “nurdles,” are the raw materials used in the manufacturing of virtually all plastic products. During transport and handling, these pellets frequently spill and escape into the environment, contributing to microplastic pollution. Moreover, microplastic powders are used in applications such as air-blasting technology, where they are employed to clean surfaces in industries ranging from ship maintenance to the aerospace sector.

Secondary microplastics are formed when larger pieces of plastic debris break down into smaller fragments due to environmental exposure. This degradation occurs through a combination of physical, chemical, and biological processes. Sunlight, particularly ultraviolet (UV) radiation, weakens the chemical bonds in plastics through photodegradation. Simultaneously, mechanical forces from, for example, wave action, wind, and abrasion with sediments further fragment plastic objects. Biodegradation by microorganisms also plays a role in the breakdown process.

Discarded plastic bags, beverage bottles, food wrappers, and fishing gear that enter the natural environment are gradually reduced to microplastic fragments. As plastic items deteriorate, they create an ever-growing supply of secondary microplastics that can persist in ecosystems for centuries.

In addition to intentional manufacturing and degradation processes, microplastics enter the environment through a myriad of everyday human activities. The laundering of synthetic textiles, such as polyester, nylon, and acrylic, releases large quantities of microfibers into wastewater. A single load of laundry can release hundreds of thousands of fibers, many of which evade conventional wastewater treatment processes and eventually reach waterways.

Microplastics are now found in virtually every ecosystem on Earth. Their environmental impacts are profound, affecting the stability of marine, freshwater, terrestrial, and even atmospheric systems. These impacts ripple across food webs, alter habitats, and contribute to a growing ecological imbalance.

The world's oceans are among the most heavily affected by microplastic pollution. Microplastics have been detected from surface waters to the deepest ocean trenches, including areas once thought to be pristine. Marine organisms at all trophic levels are susceptible to ingesting microplastics. Zooplankton, the foundation of the marine food web, often mistake microplastics for food. Ingesting these particles can reduce feeding efficiency, stifle growth, and impair reproduction.

Higher trophic level organisms, including fish, sea turtles, seabirds, and marine mammals, are also at risk. Ingestion can cause internal injuries, blockages, and a false sense of satiation, leading to starvation. Moreover, microplastics absorb and concentrate toxic chemicals such as heavy metals, persistent organic pollutants (POPs), and hydrocarbons. These contaminants can bioaccumulate in the tissues of marine organisms and biomagnify as they move up the food chain, posing serious risks to predators, including humans.

One example of marine microplastic pollution is the Great Pacific Garbage Patch, a massive accumulation of floating debris in the North Pacific Ocean. Although often characterized by large visible pieces of plastic, much of its mass consists of microplastics suspended in the water column, invisible to the naked eye but devastating to marine life.

Freshwater environments, including rivers, lakes, and wetlands, are not immune to microplastic contamination. Rivers serve as major conduits for transporting microplastics from land-based sources to oceans. Studies have found significant concentrations of microplastics in freshwater species such as fish, crustaceans, and insects.

In freshwater systems, microplastics can alter feeding behavior, inhibit growth, and affect reproductive success in aquatic organisms. The disruption of freshwater food webs can have cascading effects, diminishing biodiversity and impairing ecosystem services such as water purification and nutrient cycling.

Moreover, freshwater environments often serve as drinking water sources for human populations. The presence of microplastics in these water bodies raises concerns about the potential for contamination of human water supplies.

As microplastics proliferate in the environment, human exposure has become increasingly unavoidable. Microplastics have been detected in drinking water, food products, indoor and outdoor air, and even human tissues.

One of the primary pathways for human exposure to microplastics is through ingestion. Numerous studies have found microplastics in seafood, particularly shellfish and small fish species that are consumed whole. Filter-feeding organisms such as mussels and oysters are especially prone to accumulating microplastics, which then transfer to humans upon consumption.

Microplastics have also been identified in bottled and tap water, table salt, beer, honey, and a variety of processed foods. It has been estimated that the average person may ingest between 39,000 and 52,000 microplastic particles annually, with additional exposure through drinking water potentially doubling these figures.

Laboratory studies have shown that microplastic particles can cause physical damage to cells, including inflammation, oxidative stress, and cell death. Furthermore, the chemicals associated with plastics-such as plasticizers, flame retardants, and additives—can leach out and exert toxic effects. Microplastics may also act as vectors for harmful environmental contaminants and pathogens, introducing additional health risks. Microplastics have been identified in human tissues including brain, placenta, breast milk, and testes.

Airborne microplastics represent another significant route of human exposure. Indoor environments can harbor high concentrations of microplastic fibers shed from synthetic textiles, furniture, and household dust.

Inhaled microplastics can deposit in the respiratory tract, where they may cause mechanical irritation, inflammation, and potentially contribute to respiratory diseases. Occupational studies in industries such as textile manufacturing and plastic production have documented higher incidences of respiratory conditions among workers exposed to high levels of airborne microplastics.

Long-term exposure could also raise the risk of developing chronic respiratory diseases, including asthma, pulmonary fibrosis, and even cancer.

Dermal exposure to microplastics through skin contact is also a concern. Personal care products containing microplastics, such as exfoliating cleansers and body scrubs, bring microplastic particles into direct contact with the skin. Although the skin serves as an effective barrier against large particles, nanoparticles-extremely small microplastic fragments—can penetrate into, and through, deeper layers of the skin, especially if the barrier is compromised.

Beyond environmental and health concerns, microplastics exert significant economic burdens across multiple sectors. Their pervasive presence imposes costs on fisheries and aquaculture, tourism, waste management systems, and broader public health services.

Marine fisheries and aquaculture industries are particularly vulnerable to the impacts of microplastic pollution. Microplastic ingestion by fish and shellfish can lead to reduced growth rates, reproductive failure, and higher mortality rates, all of which diminish the productivity and profitability of fisheries.

Microplastics also strain waste management and municipal systems. Upgrading wastewater treatment facilities to better capture microplastic fibers and particles requires substantial investment. Traditional treatment plants are often ineffective at removing the smallest particles, necessitating the adoption of advanced filtration technologies.

Biofilms are initiated when free-floating, planktonic bacteria anchor to a biologic or inert surface such as an indwelling medical device. The attached bacteria multiply and progress from a state of monolayer to a microcolony and then to a critical mass, at which point bacterial crosstalk occurs, triggering a phenomenon known as quorum sensing that leads to the biofilm phenotype. Quorum sensing can turn on biofilm-producing genes that are not expressed in planktonic bacteria. The bacteria respond collectively to express factors that are specific to the biofilm phenotype, which leads to the secretion of an exopolysaccharide (EPS) matrix, definitive of the biofilm phenotype.

The biofilm phenotype is characterized morphologically by the formation of microbial towers, which are composed of layers of embedded, live bacteria with intervening water channels. Under the right environmental conditions, free-floating bacteria are released from the biofilms, and the cycle is continued at other surfaces.

### BRIEF SUMMARY OF THE INVENTION

The subject invention provides compositions and methods for detecting and/or removing microplastics, and/or other substances, from fluids, biological systems and other environments.

In preferred embodiments, the compositions of the subject invention comprise bacterial biofilm that strongly adheres to microplastics to form an aggregate thereby facilitating the detection of microplastics and/or the removal of microplastics. Microplastics may be removed from, for example, the environment, a fluid or a biological system, including cells and tissues. The biofilm can comprise living and/or inactivated bacterial or fungal cells.

In one embodiment, the biofilm composition of the subject invention is administered to the digestive tract of an animal. The composition can be formulated as, for example, a food, beverage, probiotic, or supplement.

Advantageously, it has been determined that, in accordance with the subject invention, biofilm physically associates with microplastic particles released during gum chewing. Further, exposure of biofilm-bound nanoplastics to saliva, gastric, intestinal, and colonic fluids showed ~98% retention of bound nanoplastics compared with untreated controls. This shows the stability of the biofilm binding matrix through gastrointestinal digestion.

In other embodiments, a fluid stream is contacted with a biofilm composition of the subject invention. The fluid stream may be a liquid or a gas. Examples of liquids include blood; wastewater; surface water; ground water; drinking water; fluids, such as saline, used in healthcare; water vapor; industrial effluents; and water used in industry and agriculture.

Gases that can be treated in accordance with the subject invention include, but are not limited to, ambient air, gases produced by ventilators, conditioned air, and gases used in industrial processes.

In certain embodiments, biofilm used according to the subject invention can be associated with a matrix over, and/or through, which a microplastic-containing fluid is passed. The matrix can be porous and made from, for example, a polymer, ceramic, or activated carbon.

In one embodiment, the subject invention provides a device for removing microplastics from a fluid. The device can comprise a conduit through which the fluid is passed wherein the interior of the conduit comprises a biofilm that the fluid contacts as it passes through the conduit. In one embodiment, the fluid contains cells having intracellular plastic and/or other substances and/or particles the removal of which from the cells is desired. The biofilm can be associated with a matrix that, in certain embodiments, can be disposed of and replaced once it has been contacted with the fluid.

In another embodiment, biofilms can be used to concentrate microplastics and/or other substances in order to, for example, facilitate and/or improve detection, isolation, and/or quantification of microplastics and/or other substances.

The biofilm/microplastic aggregates can be disposed of by, for example, landfilling, incineration, recycling, and enzymatic or microbial degradation.

In accordance with the subject invention, it has been found that biofilm can be used to create a strong trans-epithelial binding gradient to facilitate active transport of materials out of a cell thereby removing intracellular substances from cells. In one embodiment, this active transport mechanism can remove unwanted substances, such as microplastics from inside cells. In another embodiment, this transport mechanism can be used to export, and optionally isolate, desired components from cells.

The transepithelial barrier gradient enables the removal of internalized materials by creating a high-affinity extracellular sink. The same surface-driven forces that draw nanoplastics out of epithelial cells can also be used to transport other intracellular substances. These other substances can be, for example, heavy metals, persistent organic pollutants, or inflammatory microparticles, that accumulate within human tissues. In other embodiments, the substances are intracellular beneficial substances that are desired to be transported out of a cell to facilitate isolation and/or further use or processing.

Thus, biofilm can be used to actively transport substances out of cells in addition to blocking entry into cells. In a specific embodiment, biofilms can decrease intracellular micro- or nanoplastic particles by inducing recycling or exocytic export from endosomal compartments toward the outside of the cell.

In accordance with the subject invention, fluorescent cell imaging, AFM topology, test tube nanoplastics binding assays, gastric stability assays, and nematode clearance assays, show that biofilm can be used to block micro- and nanoplastics from reaching the epithelium and also reverse intracellular plastic burdens by facilitating export from cells into an external, high-affinity sink.

In one embodiment, compositions of the subject invention act as cleansing agents, removing unwanted materials from tissues, including intracellular materials in the cells of the tissues. In specific embodiments, the unwanted materials are hydrophobic. The tissues may be, for example, digestive tract, respiratory tract, blood, skin or hair.

In further embodiments, the subject invention provides lotions and soaps formulated with biofilm wherein the lotion can block entry of microplastics into, and through, the skin and the soap can be used to remove microplastics from skin or other surfaces. In some embodiments, the soap can be used to remove intracellular microplastics and/or other intracellular toxins from cells of the skin.

In further embodiments, the subject invention provides adjuvant materials and methods including engineered dietary fibers that can bind micro- and nanoplastics, materials that intercept aerosolized plastics before they embed in lung tissue, and surface treatments to capture plastics in food and water prior to ingestion.

Additional embodiments also provide diagnostic platforms to measure microplastic clearance, thereby tracking plastic reduction in an environment, such as the body, as well as tools to assess and monitor plastic exposure within home environments. Together, these embodiments provide comprehensive systems that, for example, protect the gut, the lungs, the skin, and environments external to the body, thereby creating a multilayered defense.

By coupling natural biofilm surface properties with binding specificity, a next generation of microbiome-inspired barrier technologies that can neutralize contaminants already within the body is provided. In this way, the transepithelial barrier gradient is a new platform for detoxification, offering a practical, biologically-grounded, safe solution to the rising tide of environmental exposures.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 is a photo simulating the microplastics binding capacity by heat inactivated Lactobacillus biofilm designated Qi601. The left-hand panel is congo red, which acts as a surrogate for microplastics. The middle panel shows red pellet at the bottom the tube demonstrating the ability of Qi601 to form an aggregate with the microplastic surrogate. The panel on the right is the resulting supernatant after treatment with Qi 601 demonstrating about 90% reduction of the microplastic surrogate (Congo red) as measured by spectrophotometry

FIG. 2 shows Qi601 biofilm associations with fluorescent microplastics when Qi601 is mixed with a liquid containing the microplastics. The light green ‘amoeboid’ shapes illustrates Qi 601 aggregates measuring about 150 micrometers, and the bright green fluorescent spheres are 10 micrometer microplastic fluorescent spheres (MPFS) adhering to the Q 601 aggregates. Where there is a lack of Qi601 aggregates there is also an absence of the fluorescent microplastic spheres, illustrating a preference of microplastic particles attaching to Qi 601 to form aggregates. The 10 micrometer microplastic particles were suspended in PBS in a 4:1 ratio (Qi601:MPFS) fluorescent microparticles, immediately placed on a microscope slide and viewed with the 100× magnification, and illuminated with fluorescent light and white light and equal intensity.

FIG. 3 shows 100 nanometer microplastic fluorescent spheres (MPFS) in PBS solution was added to Qi 601 in PBS solution in a 1:10 ratio (MPFS:Qi601), immediately placed on a slide illuminated with fluorescent light and photographed with 100× magnification. This photograph illustrates the Qi601 aggregate that measures about 100 micrometers diameter of the largest particle centrally, lightly dusted with 100 nanometer micro plastic fluorescent spheres. The individual 100 nanometer MPFS are below the level of resolution. Notable in this photograph is the absence of the microplastic fluorescent spheres in areas where there is not Qi601 indicating that there is high affinity of the nano sized fluorescent microparticles to the Qi 601 aggregates,

FIG. 4 shows spectro photometric readings of the resulting supernatant of microplastic fluorescent spheres following treatment with increasing concentrations of Qi 601 (horizontal axis) and the percentage of MPFS removed at the top of the columns associated with the increasing Qi601 concentrations. The bar chart illustrates increasing percent removal of the microplastics from the initial suspension of microplastics with increasing concentration of Qi601.

FIG. 5 shows fluorescence microscopy of Qi601 aggregates bound to microplastics. Qi601 aggregates approximately 200 μm diameter (shaded area under the “hand”) are coated with multiple, green multiple fluorescently labeled 10 μm polystyrene spheres, demonstrating selective sequestration of plastic particles to Qi601 surface.

FIG. 6 shows fluorescence microscopy of nanoplastic binding with and without Qi601. Qi601 diverts nanoplastics away from human colon epithelial cells, capturing them on its biofilm surface to prevent cellular binding. Composite fluorescence and bright-field microscopy images of human colon epithelial cells incubated with green fluorescent polystyrene nanoplastic particles are seen above Top panels (negative control) indicate that without Qi601, nanoplastics (green color) adhere extensively to the surface of the human intestinal cells, forming a continuous fluorescent layer blanketed across the cells. Bottom panels (with Qi601) show that when Qi601 is introduced, fluorescent particles are redirected to and clustered upon the Qi601 aggregates (indicated by arrows), leaving the surrounding cells largely free of bound plastics. This shift demonstrates Qi601's high-affinity sequestration mechanism-capturing micro- and nanoplastic particles before they can attach to epithelial cells and thereby functioning as a protective biofilm shield at the human gut interface.

FIG. 7 shows test tube fluorescent nanoplastic assay. One hundred nanometer, fluorescent nanoplastic suspensions glow vividly on the right test tube when Qi601 is absent. When Qi601 is added, the green fluorescence particles are captured by Qi601, sink to the bottom of the tube and disappear from the liquid above—a striking image of sequestration in action.

FIG. 8 shows Atomic Force Microscopy (AFM) of Qi601 Aggregates. AFM imaging of Qi601 aggregates reveals their characteristic cobblestone-like surface topology at the micron and nanometer scales. Each individual aggregate, roughly 150-300 μm in diameter, is composed of densely packed micro-domains formed from the self-assembled aggregates of Qi601 (blue arrows). High-resolution AFM scans show an uneven, biofilm-like matrix with deep interstitial grooves and crevices capable of physically entrapping nanoplastics. When nanoplastics are introduced into the imaging field, they are observed adhering within these depressions and along ridges of the Qi601 surface—a direct visualization of the material's physical binding behavior (red arrows). This topographic architecture explains the strong sequestration performance seen in fluid assays and cell models: the irregular biofilm terrain dramatically increases available surface area and creates nanoscale pockets of high adhesive potential. These AFM findings confirm that Qi601's mechanism of action is significantly physical—a biofilm-derived matrix that captures, immobilizes, and retains nanoplastics through texture, topology, and surface chemistry rather than chemical degradation or metabolic interaction.

FIG. 9 shows In Vivo Evidence of Nanoplastic Clearance by Qi601 in C. elegans. Fluorescence microscopy of C. elegans nematodes fed bright green fluorescent nanoplastics shows intense intestinal fluorescence in controls (top panel) indicating epithelial accumulation. Worms co-fed Qi601 (bottom) display markedly reduced gut fluorescence, with bright green nanoparticles appearing in excreta (green arrows). The dull green coloring in the bottom panel is the result of autofluorescence of the C. elegans themselves. These results demonstrate that Qi601 binds the ingested bright green nanoplastics, preventing their adhesion to intestinal surfaces, and promotes natural elimination through the digestive tract.

FIG. 10 shows Qi601 Facilitates Removal of Intracellular Nanoplastics from Human Colon Cells. Bright-field and fluorescence microscopy of human colon cells (Caco2) show Qi601's detoxification effects. In untreated cells (left panels), 100 nanometer fluorescent nanoplastic particles (green spheres) accumulate throughout the cytoplasm, forming dense intracellular clusters. After 24 hours of exposure to nanoplastics followed by 4 hours of Qi601 treatment (right panels), intracellular fluorescence was markedly reduced, indicating that nanoplastics were drawn out and sequestered by Qi601 aggregates. These findings demonstrate that Qi601 not only prevents nanoplastic entry into epithelial cells but may also facilitate the removal of internalized particles—a novel, dual-action detoxification mechanism.

FIG. 11 shows Nanoplastic Internalization in Human Colon Cells Without Qi601 Protection. Fluorescence microscopy of human colon cells exposed for 24 hours to 100 nanometer fluorescent polystyrene (PS) nanoplastic spheres (green) demonstrates active cellular uptake and intracellular accumulation. The upper left panel illustrates the appearance and scale with diffuse “dust-like” distribution of the 100 nanometer nanoplastics on Qi601. In stark contrast, the main composite images at the right upper and lower quadrants show bright punctate fluorescence within the cytoplasm and along nuclear boundaries, indicating internalization of thousands of nanoparticles grouped into spheres. Magnified views reveal nanoplastic localization in the perinuclear region and within the cytoplasm, suggesting uptake into endosomal or lysosomal compartments. These findings establish the baseline condition in which nanoplastics penetrate and persist inside human colon cells, forming dense intracellular clusters in the absence of Qi601 protection.

FIG. 12 shows that Qi601 Reduces Intracellular Nanoplastics in Human Colon Cells After 24 Hour Exposure. Fluorescence microscopy of human colonic epithelial cells demonstrates the detoxifying effects of Qi601 following nanoplastic exposure, In untreated controls (left panels), cells exposed for 24 hours to 100 nanometer fluorescent polystyrene nanoplastic spheres (green) show dense cytoplasmic and perinuclear accumulation, indicating active internalization via endosomal pathways. Magnified views in the bottom left highlight tight clustering of nanoparticles near the nucleus. In contrast, Qi601-treated cells (right panels)—after identical 24-hour nanoplastic exposure followed by 4 hours of Qi601 treatment—display markedly reduced intracellular fluorescence. Most nanoplastic particles have been cleared or redistributed to extracellular Qi601 aggregates (magenta), leaving only faint residual signals or a fine particulate “dusting.” These findings indicate that Qi601 may function as a post-exposure detoxification agent, actively extracting internalized nanoplastics from human colon cells and reducing intracellular burden.

FIG. 13 shows Reduction of Intracellular Nanoplastics Following Qi601 Treatment—Quantitative fluorescence analysis of human colon epithelial cells demonstrates that treatment with Qi601 significantly decreases intracellular nanoplastic (NP) accumulation. Cells exposed to 100 nanometer fluorescent polystyrene nanoplastics for 24 hours were subsequently treated with 3 mg/mL Qi601 exhibited an 18% reduction in intracellular fluorescence intensity compared to untreated controls (p=0.01). This statistically significant decrease supports the hypothesis that Qi601 facilitates removal of internalized nanoplastics via a transepithelial barrier gradient mechanism.

FIG. 14 is a scanning electron microscopic picture of Qi601 wrapping around 10 micron plastic spheres.

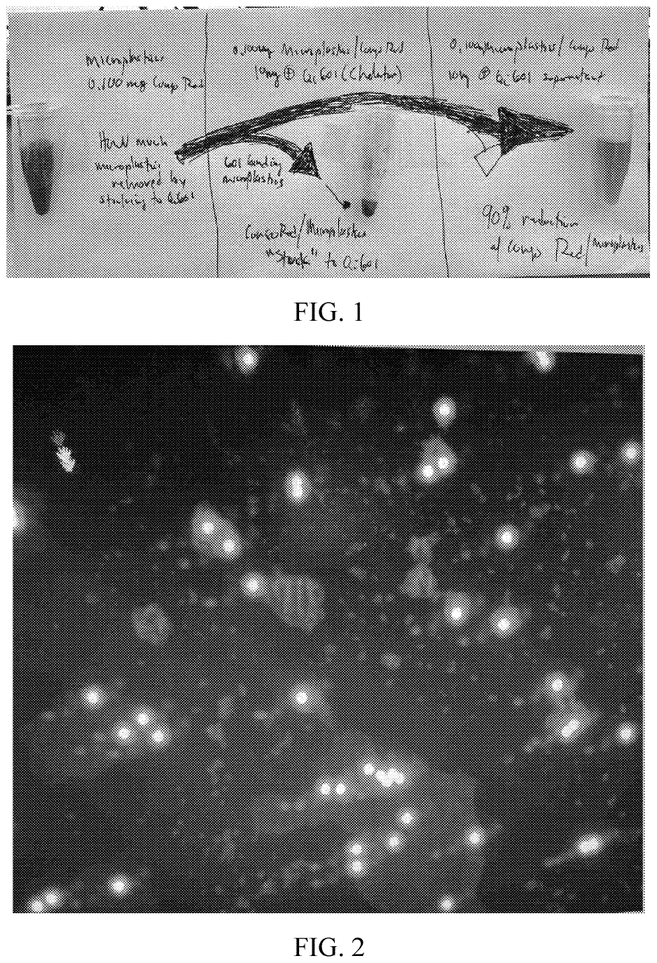

### DETAILED DESCRIPTION OF THE INVENTION

The subject invention provides compositions and methods for detecting and/or removing microplastics, and/or other substances, from fluids, biological systems and other environments.

In preferred embodiments, the compositions of the subject invention comprise bacterial biofilm that strongly adheres to microplastics to form an aggregate thereby facilitating the detection of microplastics and/or the removal of microplastics. Microplastics may be removed from, for example, the environment, a fluid or a biological system, including cells and tissues. The biofilm can comprise living and/or inactivated bacterial or fungal cells.

In one embodiment, the biofilm composition of the subject invention is administered to the digestive tract of an animal. The composition can be formulated as, for example, a food, beverage, probiotic, or supplement.

In other embodiments, a fluid stream is contacted with a biofilm composition of the subject invention. The fluid stream may be a liquid or a gas. Examples of liquids include blood; wastewater; surface water; ground water; drinking water; fluids, such as saline, used in healthcare; water vapor; industrial effluents; and water used in industry and agriculture.

Gases that can be treated in accordance with the subject invention include, but are not limited to, ambient air, gases produced by ventilators, conditioned air, and gases used in industrial processes.

In certain embodiments, biofilm used according to the subject invention can be associated with a matrix over, and/or through, which a microplastic-containing fluid is passed. The matrix can be porous and made from, for example, a polymer, ceramic, or activated carbon.

In one embodiment, the subject invention provides a device for removing microplastics from a fluid. The device can comprise a conduit through which the fluid is passed wherein the interior of the conduit comprises a biofilm that the fluid contacts as it passes through the conduit. In one embodiment, the fluid contains cells having intracellular plastic and/or other substances and/or particles the removal of which from the cells is desired. The biofilm can be associated with a matrix that, in certain embodiments, can be disposed of and replaced once it has been contacted with the fluid.

In another embodiment, biofilms can be used to concentrate microplastics and/or other substances in order to, for example, facilitate and/or improve detection, isolation, and/or quantification of microplastics and/or other substances.

The biofilm/microplastic aggregates can be disposed of by, for example, landfilling, incineration, recycling, and enzymatic or microbial degradation.

Nanoplastics can enter human epithelial cells through multiple pathways, such as passive diffusion across the cell wall and clathrin-mediated endocytosis. The microplastics can then become trapped within intracellular vesicles, such as endosomes and lysosomes.

In accordance with the subject invention, it has been found that biofilm can be used to create a strong trans-epithelial binding gradient to facilitate active transport of materials out of a cell thereby removing intracellular substances from cells. In one embodiment, this active transport mechanism can remove unwanted substance, such as microplastics from inside cells. In another embodiment, this transport mechanism can be used to export, and optionally isolate, desired components from cells. In specific embodiments, the substance transported out of the cell is hydrophobic.

The transepithelial barrier gradient enables the removal of internalized materials by creating a high-affinity extracellular sink. The same surface-driven forces that draw microplastics out of epithelial cells can also be used to transport other intracellular substances. These other substances can be, for example, heavy metals, persistent organic pollutants, or inflammatory microparticles, that similarly accumulate within human tissues. In other embodiments, the substances are intracellular beneficial substances that are desired to be transported out of a cell to facilitate isolation and/or further use or processing.

In this model, the aggregates of Qi601 outside the cell act as a molecular magnet, “pulling” nanoplastics back through the membrane and/or facilitating their release via natural vesicular recycling and exocytic export.

Fluorescence microscopy and time-lapse imaging show declining intracellular fluorescence after Qi601 exposure establishes that biofilm can not only block new entry but also draw existing plastics out of cells, including human cells.

Thus, biofilm can be used to actively transport substances out of cells in addition to blocking entry into cells. In a specific embodiment, biofilms can decrease intracellular micro- or nanoplastic particles by inducing recycling or exocytic export from endosomal compartments toward the outside of the cell.

In accordance with the subject invention, fluorescent cell imaging, AFM topology, test tube microplastics binding assays, gastric stability assays, and nematode clearance assays, show that biofilm can be used to block micro- and nanoplastics from reaching the epithelium and also reverse intracellular plastic burdens by facilitating export from cells into an external, high-affinity sink.

Fluorescence microscopy and quantitative analysis show that Qi601 facilitates the removal of intracellular nanoplastics (NPs) from human colon epithelial cells through a mechanism likely driven by a transepithelial barrier gradient.

In untreated conditions, cells internalize fluorescent nanoplastic particles, which persist within the cytoplasm and perinuclear regions, maintaining a steady intracellular burden; however, upon addition of Qi601 following exposure, intracellular fluorescence intensity decreases significantly, consistent showing that extracellular Qi601 aggregates act as an extracellular high-affinity sink. This creates a binding gradient that favors the outward movement of nanoplastics, through for example, vesicular recycling, endosomal export, and/or passive efflux, toward the extracellular Qi601 biofilm surface where nanoplastics are bound, sequestered and prevented from reentry.

This gradient-driven clearance mechanism reflects the same physical and electrostatic binding principles that enable biofilm to prevent plastic attachment in the first place, thus uniting prevention and detoxification within a single mechanistic framework. Thus, biofilm not only intercepts nanoplastics before they enter cells but can also reverse intracellular accumulation, transforming the epithelial interface from a passive barrier into an active detoxification system. Extracellular biofilm acts as a high-affinity sink, creating a concentration gradient that promotes outward transport of intracellular NPs through, for example, vesicular recycling, endosomal export, and/or passive efflux.

In one embodiment, it was found that by using the compositions and methods of the subject invention to remove plastics from cells that have been exposed for 24 hours to nanoplastics, it is possible to significantly improve the cell survival. This was measured by cellular protein content, which was used to normalize other parameters. Use of biofilm can, therefore, be used according to the subject invention to improve mitochondrial functioning thereby improving cell function and survival as well as treating pathologies associated with compromised mitochondrial functioning, particularly due to toxicity.

The substance to be removed can also be a substance that is generated by the cell but is insufficiently removed by the cell, such as in the case of lysosome storage diseases. Thus, to treat a lysosome storage disease, such as Tay-Sachs disease, cells can be contacted with a biofilm to facilitate removal from the cell of the pathologically accumulating material. The biofilm may have live cells, deactivated cells, or only the extracellular material (e.g., exopolysaccharides). In one embodiment, the subject has been diagnosed with a lysosome storage disease before the administration of the biofilm and/or the progress/status of the disease is monitored during and/or after treatment.

In one embodiment, compositions of the subject invention act as cleansing agents, removing unwanted materials from tissues, including intracellular materials in the cells of the tissues. In specific embodiments, the unwanted materials are hydrophobic. The tissues may be, for example, digestive tract, respiratory tract, blood, skin, or hair.

In further embodiments, the subject invention provides lotions and soaps formulated with biofilm wherein the lotion can block entry of microplastics into, and through, the skin and the soap can be used to remove microplastics from skin or other surfaces. In some embodiments, the soap can be used to remove intracellular microplastics and/or other intracellular toxins from cells of the skin.

In further embodiments, the subject invention provides adjuvant materials and methods including engineered dietary fibers that can bind micro- and nanoplastics, materials that intercept aerosolized plastics before they embed in lung tissue, and surface treatments to capture plastics in food and water prior to ingestion.

Additional embodiments also provide diagnostic platforms to measure microplastic clearance, thereby tracking plastic reduction in an environment, such as the body, as well as tools to assess and monitor plastic exposure within home environments. Together, these embodiments provide comprehensive systems that, for example, protect the gut, the lungs, the skin, and environments external to the body, thereby creating a multilayered defense.

By coupling natural biofilm surface properties with binding specificity, a next generation of microbiome-inspired barrier technologies that can neutralize contaminants already within the body is provided. In this way, the transepithelial barrier gradient is a new platform for detoxification, offering a practical, biologically-grounded, safe solution to the rising tide of environmental exposures.

In specific embodiments, the compositions comprise a strain of Lactobacillus fermentum bacterium grown as biofilm, and the use thereof. The bacteria may be live or inactivated (dead). The subject invention also provides compositions comprising L. fermentum bacterium in a lyophilized, freeze dried, and/or lysate form.

The convergence of barrier protection and intracellular clearance according to the subject invention demonstrates how microbiology-inspired design can be translated into tangible human protection such that the body is no longer merely a passive recipient of environmental contaminants but can engage mechanisms for active participation in their removal. Biofilm is bio-interactive, forming a surface that prevents micro- and nanoplastics from adhering to or penetrating cells while also facilitating their removal once internalized. This mechanism marks a paradigm shift in the biological handling of pollutants. By coupling these innate transport routes with an external, high-affinity sequestration platform, biofilm transforms the epithelial barrier from a passive boundary into an active detoxification interface.

All embodiments of the subject invention include embodiments comprising a step whereby after, or during, the treatment, the subject, tissue, cell, and/or environment is examined to determine whether a change, such as reduced unwanted material, and/or improved health, or isolation of desired material has occurred as a result of the treatment.

### SELECTED DEFINITIONS

As used herein, reference to an “isolated” microbe refers to one that has been removed from materials with which it exists in nature. The microbe may be isolated from, for example, soil, blood, mucous, or milk such that it is removed from, and is no longer mixed or otherwise associated with, those materials to the extent that it is in nature. Such isolation can be used to impart upon the microbe markedly different characteristics, such as the production of different, or different amounts of, compounds, compared to what the microbe exhibits in its natural state.

As used here in, a “biologically pure culture” is one that has been isolated from other biologically active materials, including any materials with which it may have been associated in nature. In a preferred embodiment, the culture has been isolated from all other living cells. In further preferred embodiments, the biologically pure culture has advantageous characteristics compared to a culture of the same microbial species that may exist in nature. The advantageous characteristics can be, for example, enhanced production of one or more desirable growth by-products.

As used herein, an “isolated” or “purified” compound is substantially free of other compounds, such as cellular material, with which it is associated in nature. A purified or isolated polynucleotide (ribonucleic acid (RNA) or deoxyribonucleic acid (DNA)) is free of sequences that flank it in its naturally-occurring state. A purified or isolated polypeptide is free of, for example, other cellular material with which it would be associated in nature. “Isolated” in the context of a microbial strain means that the strain is removed from the environment in which it exists in nature. Thus, the isolated strain may exist as, for example, a biologically pure culture, or as spores (or other forms of the strain) in association with a carrier.

As provided herein, “pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or adjuvant” refers to an excipient, carrier or adjuvant that can be administered to a subject, together with an active ingredient, and which does not destroy the pharmacological activity thereof and is nontoxic when administered in doses sufficient to deliver a therapeutic amount of the compositions provided herein. Except insofar as any conventional media or agent is incompatible with the active ingredient, its use in the compositions of the invention is contemplated. Supplementary active ingredients can also be incorporated into the compositions.

Examples of carriers suitable for use in the compositions are known in the art and such embodiments are within the purview of the invention. The acceptable carriers and excipients, include, but are not limited to, aqueous vehicles, water-miscible vehicles, non-aqueous vehicles, stabilizers, solubility enhancers, isotonic agents, buffering agents, suspending and dispersing agents, wetting or emulsifying agents, complexing agents, sequestering or chelating agents, cryoprotectants, lyoprotectants, thickening agents, pH adjusting agents, and inert gases. Other suitable excipients or carriers include, but are not limited to, dextran, glucose, maltose, sorbitol, xylitol, fructose, sucrose, and trehalose.

As used herein, “microplastics” refer to pieces of plastic that are less than 5 mm in length. Unless the context clearly dictates otherwise, as used herein “microplastics” include nano-plastics.

“Food-grade bacteria” refers to any bacteria, alive or dead, that have no harmful effect on human health or that have a GRAS (generally recognized as safe) status, or a similar designation in European or other jurisdictions. The term “probiotic” refers to food-grade bacteria which perform beneficial functions to subject organisms when they are present in the subject organisms.

“Planktonic” refers to a phenotype typical to microorganisms (bacteria, fungi, and/or protozoa, with associated bacteriophages and other viruses) that float freely in a liquid medium.

As used herein, a “biofilm” is a complex grouping of microorganisms, such as bacteria, wherein the cells adhere to each other using a matrix usually composed of, but not limited to, polysaccharide material. The cells in biofilms have physiologically distinct properties compared to planktonic cells of the same organism, which are single cells that can float or swim in liquid or gaseous mediums, or reside on or in solid or semi-solid surfaces.

For the purpose of the present invention the abbreviation cfu shall designate a “colony forming unit” that is defined as the number of bacterial cells as revealed by microbiological counts on agar plates.

The term “treatment” or any grammatical variation thereof (e.g., treat, treating, etc.), as used herein, includes but is not limited to, the application or administration to a subject (or application or administration to a cell or tissue from a subject) with the purpose of delaying, slowing, stabilizing, curing, healing, alleviating, relieving, altering, remedying, less worsening, ameliorating, improving, or affecting ta condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical or mental well-being.

The term “prevention” or any grammatical variation thereof (e.g., prevent, preventing, etc.), as used herein, includes but is not limited to, at least the reduction of likelihood of the risk of (or susceptibility to) an event occurring (e.g., reducing the likelihood of microplastics moving from a digestive tract into blood circulation or other tissues, or causing at least one of the clinical symptoms of a condition not to develop in a patient that may be susceptible to the condition but does not yet experience or display symptoms of the condition). The term “prevention” refers to avoiding, delaying, forestalling, or minimizing one or more unwanted features associated with an unwanted event or feature, and/or completely or almost completely preventing the unwanted event and/or its symptoms altogether. Prevention can further include, but does not require, absolute or complete prevention, meaning a condition may still develop, or an event may still occur, at a later time and/or with a lesser severity than it would without preventative measures. Prevention can include reducing the severity of the onset of a condition, event, disease or disorder, and/or inhibiting the progression thereof.

As used herein, the term “subject” refers to an animal. The animal may be, for example, a human, pig, horse, goat, cat, mouse, rat, dog, ape, fish, chimpanzee, orangutan, guinea pig, hamster, cow, sheep, bird (including chicken), as well as any other vertebrate or invertebrate. The preferred subject, in the context of this invention, is a human of any age and/or gender.

Administration of the compositions provided herein is preferably in “an effective amount,” this being an amount sufficient to result in a desired physical, biological or medical response of a cell, tissue, system, animal, or human that is being treated. The actual amount administered, and the rate and time-course of administration, will depend on the circumstances, as well as the subject.

Ranges provided herein are understood to be shorthand for all of the values within the range. For example, a range of 1 to 20 is understood to include any number, combination of numbers, or sub-range from the group consisting of 1, 2, 3, 4, 5, 6, 7, 8, 9, 10, 11, 12, 13, 14, 15, 16, 17, 18, 19, 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, “reduce” refers to a negative alteration, and the term “increase” refers to a positive alteration, each of at least 1%, 5%, 10%, 25%, 50%, 75%, or 100%.

As used herein, “reference” refers to a standard or control event or condition.

The transitional term “comprising,” which is synonymous with “including,” or “containing,” is inclusive or open-ended and does not exclude additional elements or method steps not recited. By contrast, the transitional phrase “consisting of” excludes any element, step, or ingredient not specified in the claim. The transitional phrase, “consisting essentially of,” limits the scope of a claim to the specified materials or steps “and those that do not materially affect the basic and novel characteristic(s)” of the claimed invention, e.g., the ability to preclude bacterial growth. Use of the term “comprising” contemplates embodiments “consisting” and “consisting essentially” of the recited component(s).

Unless specifically stated or is obvious from context, as used herein, the term “or” is understood to be inclusive. Unless specifically stated or is obvious from context, as used herein, the terms “a,” “an,” and “the” are understood to be singular or plural.

Unless specifically stated or is obvious from context, as used herein, the term “about” is understood as within a range of normal tolerance in the art, for example, within 2 standard deviations of the mean. The term “about” can be understood as within 10%, 9%, 8%, 7%, 6%, 5%, 4%, 3%, 2%, 1%, 0.5%, 0.1%, 0.05%, or 0.01% of the stated value.

Bacteria that can be grown in the biofilm phenotype according to the subject invention include the following:

TABLE 1

| Genus | Gram Stain | Notable Species/Examples |
|---|---|---|
| Bacillus | Positive | B. subtilis |
| Lactobacillus | Positive | L. plantarum; L. fermentum |
| Lactococcus | Positive | L. lactis |
| Enterococcus | Positive | E. faecalis |
| Propionibacterium | Positive | P. spp. |
| Pseudomonas | Negative | P. aeruginosa |
| Escherichia | Negative | E. coli |
| Klebsiella | Negative | K. pneumoniae |
| Acinetobacter | Negative | A. baumannii |
| Bifidobacterium | Positive | B. longum |

In specific embodiments, the bacteria may be, for example, Lactobacillus fermentum, Bifidobacterium longum, Lactobacillus acidophilus, Enterococcus faecalis, Lactobacillus paracasei, Lactobacillus plantarum, Lactobacillus rhamnosus, and/or Lactococcus lactis, used either alone or in any combination, including with other microbes.

### Lactobacillus Spp.

In some embodiments, the bacterial strain grown as biofilm for use according to the subject invention is Lactobacillus fermentum Qi6, also referred to herein as LfQi6. Lactobacillus fermentum is a Gram-positive rod. Lactobacillus fermentum Qi6 (Lf Qi6) can be grown in MRS media at 37 °C. In one embodiment, the subject invention provides a biologically pure culture of LfQi6, grown as a biofilm. Specifically taught herein are methods for inducing and identifying the biofilm phenotype. Further provided herein are methods of utilizing the biofilm phenotype. In preferred embodiments, the subject invention provides hydrophobic compositions comprising LfQi6 biofilm. LfQi6 biofilm that has been heat-inactivated is referred to herein as Qi601.

A culture of the L. fermentum microbe has been deposited with the American Type Culture Collection (ATCC), 10801 University Blvd., Manassas, Va. 20110-2209 USA. The deposit has been assigned accession number ATCC No. PTA-122195 by the repository and was deposited on Jun. 10, 2015.

The subject culture has been deposited under conditions that assure that access to the culture will be available during the pendency of this patent application to one determined by the Commissioner of Patents and Trademarks to be entitled thereto under 37 CFR 1.14 and 35 U.S.C 122. The deposit is available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the culture. The depositor acknowledges the duty to replace the deposit should the depository be unable to furnish a sample when requested, due to the condition of the deposit. All restrictions on the availability to the public of the subject culture deposit will be irrevocably removed upon the granting of a patent disclosing it.

Microbial biofilms may be grown on relatively non-porous media, such as spheres, in, for example, a rotating-tube reactor system. This non-porous media may be represented by sterile 5 mm glass spheres. The reactor vessel may be a sterile 50 ml tube polystyrene Falcon tube, or a similar column placed on a rotating shaker. The tube or column may have water-tight tops such that rotations on a mechanical rotator do not result in spillage of vessel contents (i.e., contents are secured via screw-top, rubber or cork plug). The spheres may be immobilized at one end of the tube or column by an open mesh, autoclavable plastic plug. Each tube or column may contain sufficient open space such that as the device is mechanically rotated, a flushing action is generated to provide shear force to the cellular organisms adhering to the sphere surfaces without generating vacuum or static effect upon the media within the enclosed system. Devices such as tube inlets and tube outlets circulated via filtration pumps permit media replacement and aseptic removal of individual spheres by use of sterile forceps or similar device. Circulating biological growth media may be sterilized via membrane filtration. Positive determination of adequate biofilm formation growing on spheres may be performed via various microscopic staining techniques.

Methods for growing biofilm are known in the art and are described in, for example, WO 2012/118535, which is incorporated herein, in its entirety, by reference, including the publications cited in that reference, such as those cited at pages 26-31.

Preferably, the biofilm is hydrophobic. Hydrophobicity can be measured by, for example, the well-known static contact angle assay, which uses a drop tensiometer to measure the angle of water droplets on a biofilm-coated surface. The greater the angle, the greater the hydrophobicity of the biofilm. In preferred embodiments, the angle is greater than 900, greater than 1000, greater than 110°, greater than 120°, greater than 130°, greater than 140°, or even greater than 150°.

In specific embodiments, the bacteria that are grown in the biofilm phenotype are food-grade bacteria.

In one embodiment, the present invention relates to microbial biofilms, including biofilms formulated as probiotics, capable of removing or sequestering microplastics from an environment to which the biofilm is exposed, or from a substance or a subject that contain microplastics. Substances that contain microplastics include edible compositions, such as plant-based or animal-based foods or feeds; and drinkable solutions, including water, milk, baby formula, extracts and other beverages.

Substances may also include fluids, such as saline and ventilator gases, in a healthcare environment. Substances may also include raw agricultural products used to produce foods and drinkable solutions.

As such, the present invention relates also to methods of using biofilm to prevent the uptake of microplastics by a subject, or in methods to filter microplastics out of substances prior to exposing a subject to said substances, or to remove microplastics after subjects are exposed. The environment may include an aqueous environment, such as the gastrointestinal tract of a subject, or the environment in which the subject, or its feed or food, resides, such as surface water or other environment.

The microorganisms may be any type of bacteria, fungi or yeasts capable of forming a biofilm. Administration of the biofilm to a subject may be accomplished by any method likely to introduce the biofilm to a desired location, such as the gastro-intestinal tract, of the subject. The biofilm can be mixed with a carrier and applied to liquid or solid feed or to drinking water. The biofilm can also be formulated as, for example, a pill or capsule. The carrier material should be non-toxic to the subject. When dealing with live biofilm, the carrier material should also be non-toxic to the microbe. The biofilm may also be formulated as a paste to be directly injected into a subject's mouth. The formulation may include added ingredients to improve palatability, improve shelf-life, impart nutritional benefits, and the like. The amount of biofilm to be administered is governed by factors affecting efficacy. When administered in food, feed or drinking water the dosage can be spread over a short time, or a period of days, weeks, or longer. One or more types of biofilm may be administered together.

### Applications

In one embodiment of the present invention, biofilm may be used as a preventive measure, to reduce the exposure of a subject to microplastics from, for example, consumables or environments where microplastics are present. Biofilm of the present invention may also be used to substantially reduce microplastics in a subject.

By routinely administering an effective dose to a subject, the risk of contamination by microplastics may be substantially reduced or substantially eliminated.

The biofilm may also be used, according to another embodiment of the present invention, with food-producing plants and animals. Such animals can be, for example, aquatic animals such as fish and shrimp, or insects such as honey bees, or insects used as edible food or feed. In one embodiment, biofilm of the present invention may, for example, be added to tanks and ponds containing aquatic animals, or to water or feed materials in insect rearing facilities or bee hives.

### Preparation and Administration

In some embodiments, the bacterial strain that is grown as biofilm is Lactobacillus fermentum Qi6, also referred to herein as LfQi6. In one embodiment, the subject invention provides an isolated or a biologically pure culture of LfQi6. In another embodiment, the subject invention provides a biologically pure culture of LfQi6, grown as a biofilm. Specifically taught herein are methods for inducing and identifying the biofilm phenotype. Further provided herein are methods of utilizing the biofilm phenotype.

One biofilm may be administered alone or in conjunction with a second (or more), different biofilm. Any number of different biofilms may be used in conjunction. By “in conjunction with” is meant together, substantially simultaneously or sequentially. The compositions may be administered in the form of tablet, pill, capsule, paste, powder or liquid, for example. One preferred form of application involves the preparation of an encapsulated, heat-inactivated material comprising the biofilm. Another preferred form of application involves the preparation of a lyophilized capsule of the biofilm. Still another preferred form of application involves the preparation of a heat dried capsule of the present invention. In other embodiments, the bacteria may be live. In these embodiments, the bacteria may already be in the biofilm phenotype and/or may grow into the biofilm phenotype in vivo.

A significant practical advantage is that the biofilm may be administered in a convenient manner such as by the oral, or suppository routes. Depending on the route of administration, biofilm may be required to be coated in a material to protect said material from the action of enzymes, acids and other natural conditions that may reduce the efficacy of the biofilm. For example, biofilm may be co-administered with enzyme inhibitors or in liposomes.

In the case of sterile powders for the preparation of sterile solutions, the preferred methods of preparation are vacuum-drying and the freeze-drying technique, which yield a powder of the biofilm plus any additional desired ingredient from previously sterile-filtered solution thereof. Additional preferred methods of preparation include but are not limited to lyophilization and heat-drying.

The biofilm may be orally administered, for example, with a diluent or with an edible carrier, or it may be enclosed in hard or soft shell gelatin capsule, or it may be compressed into tablets designed to pass through the stomach (i.e., enteric coated), or it may be incorporated directly with the food or feed of dietary preparations. For oral administration, the biofilm may be incorporated with excipients and used in the form of ingestible tablets, buccal tablets, troches, capsules, elixirs, suspensions, syrups, wafers, and the like.

The tablets, troches, pills, capsules, and the like, as described above, may also contain the following: a binder such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, lactose or saccharin may be added or a flavoring agent such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, food glace, sugar or both. A syrup or elixir may contain the active compound, sucrose or sugar replacements as a sweetening agent, methyl and propylparabens as preservatives, a dye and flavoring such as cherry or orange flavor. Of course, any material used in preparing any dosage unit form should be pure and substantially non-toxic in the amounts employed.

The specification for the dosage unit forms of the invention may be dictated by and may be directly depending on (a) the unique characteristics of the particular biofilm and the particular effect to be achieved, and (b) the limitations inherent in the art of compounding such biofilm.

The carrier may be in the form of milk or portions thereof including yogurt, skim milk, skim milk powder. Non-milk or non-lactose containing milk replacing products based on soy, oats, rice, almonds or the like may also be employed. The skim milk powder or above-mentioned replacements are conventionally suspended in phosphate buffered saline (PBS), autoclaved or filtered to eradicate proteinaceous and living contaminants, then freeze dried, heat dried, vacuum dried, or lyophilized.

Some other examples of substances that can serve as carriers are sugars, such as lactose, glucose and sucrose; starches such as corn starch and potato starch; cellulose and its derivatives such as sodium carboxymethycellulose, ethylcellulose and cellulose acetates; powdered tragacanth; malt; gelatin; talc; stearic acids; magnesium stearate; calcium sulfate; calcium carbonate; vegetable oils, such as peanut oils, cotton seed oil, sesame oil, olive oil, corn oil and oil of theobroma; polyols such as propylene glycol, glycerin, sorbitol, mannitol, and polyethylene glycol; agar; alginic acids; pyrogen-free water; isotonic saline; cranberry, extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, antioxidants and preservatives, can also be present.

Accordingly, the subject may be orally administered an effective amount of at least one biofilm and an acceptable carrier in accordance with the present invention. The biofilm may be selected from the bacteria listed in Table 1.

The compositions provided herein may contain a single (unit) dose of biofilm. Suitable doses of probiotic biofilm may be in the range 10⁴ to 10¹² cfu, e.g., one of 10⁴ to 10¹⁰, 10⁴ to 10⁸, 10⁶ to 10¹², 10⁶ to 10¹⁰, or 10⁶ to 10⁸ cfu. In some embodiments, doses may be administered once or twice daily. In some embodiments, a composition for use according to the present invention may comprise at least about 0.01%, about 0.05%, about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% by weight of the biofilm, such as Qi601. In some embodiments, the compositions may comprise, one of at least about 0.01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0.3% to about 5%, about 0.4% to about 5%, about 0.5% to about 5%, about 1% to 10 about 5%, by weight of the biofilm.

For inactivated biofilm the dosages may be, for example, from 1 mg to 500 mg, 2 mg to 400 mg, 5 mg to 250 mg, 8 mg to 150 mg, 10 mg to 100 mg, 12 mg to 75 mg, 15 mg to 50 mg, 18 mg to 25 mg, or about 20 mg.

### Materials and Methods

### Culture of Lf Qi6 Biofilm

After being isolated and identified, Lf Qi6 was cultured in MRS agar plate. The culture was then incubated in 5 ml of MRS broth for 24 hours at 37 °C. 1 ml of the culture was transferred into a T-150 tissue culture plate with 25 ml of MRS broth. 25 ml of MRS media was changed every 48 hours to allow the biofilm of Lf Qi6 to grow as lawn on the bottom of the culture plate. The culture was then grown for 7 days to produce a thick biofilm layer. The grown biofilm layer was subsequently scraped out and suspended in fresh medium. Freezer stacks were made with glycerol and stored in −80°.

A biofilm phenotype of Lf Qi6 in frozen stock was cultured in 10 ml of fresh MRS media for 24 hours at 37°. 10 ml of culture was inoculated into 25 L of MRS media with 500 g sterile glass wool. The biofilm was then cultured for 72 hours under static conditions at 37 °C. The culture was mixed every 24 hours with a gentle shaking, after which the media and glass wool were harvested. The biofilm cells were subsequently detached from the glass wool via sonication. The cells were further centrifuged to concentrate the biofilm of Lf Qi6, which was then suspended in sterile water. This scale-up yields a biofilm culture at a concentration of 50 g/25 L.

### EXAMPLES

### Example 1

In one embodiment Qi601 (heat-inactivated biofilm of Lactobacillus fermentum) is formulated in a capsule for oral delivery to remove microplastics from the digestive tract of an animal, preferably a human.

The capsule can have, for example, 20 mg of Qi601 formulated with microcrystalline cellulose.

The subject can ingest, for example, 1 capsule per day to remove microplastics from the digestive tract, thereby preventing or reducing systemic distribution of the microplastics.

### Example 2: Association of Microplastics with Lactobacillus Biofilm

When exposed to 10-micron green, fluorescent microspheres, the large spherical plastic particles are seen clustered along the irregular surfaces of Qi601, forming bright rings of fluorescence that delineate the Qi601's biofilm's contours. (FIG. 5).

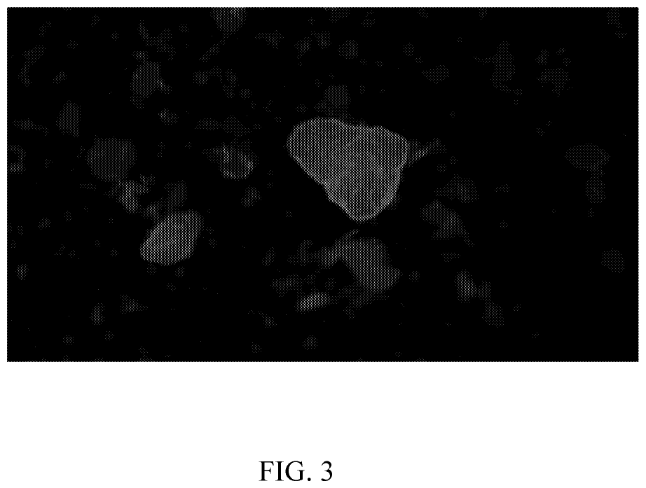

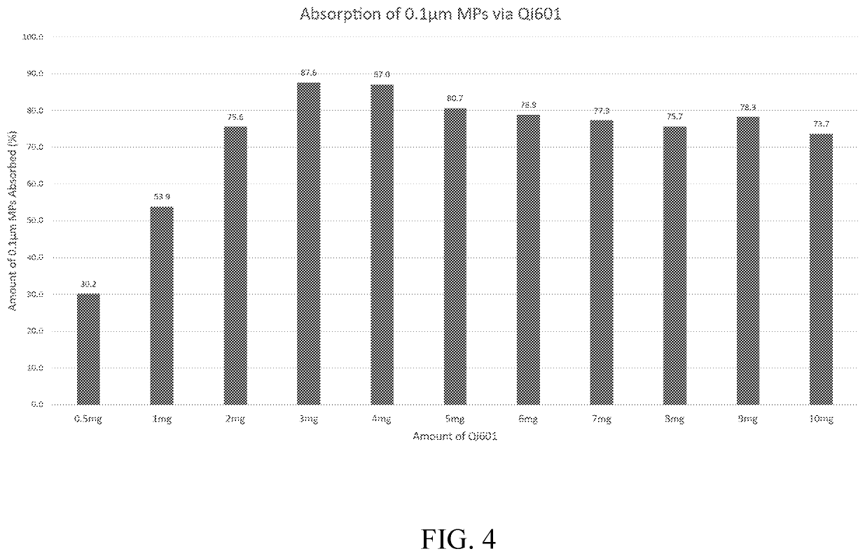

In parallel fluorescent microscopy experiments using 100-nanometer fluorescent nanoplastic spheres, the same pattern emerges, this time at the nanoscale: Qi601 aggregates act as discrete focal points of binding of 100 nanometer, fluorescent particles, dusting the surface of Qi601 biofilm aggregates. Areas of the field devoid of Qi601 remain entirely free of fluorescence (FIG. 3).

This staining pattern of both 10 micron and 100 nanometer fluorescent plastic particles confirms that Qi601 exhibits a high-affinity, size-independent attraction for plastic materials, driven by its surface topology and electrostatic properties.

The absence of bright green fluorescent particles outside the Qi601 boundaries underscores the selectivity of this interaction and provides direct visual evidence of the ability of Qi601 to physically sequester substances, including sequestering plastics of varying sizes. These findings provide visual confirmation that Qi601 functions as a physical sequestration platform, capable of attracting and retaining both micro- and nanoplastic contaminants through surface-driven adhesion.

### Example 3: Nanoplastic Binding on Human Intestinal Cells Blocked by Biofilm

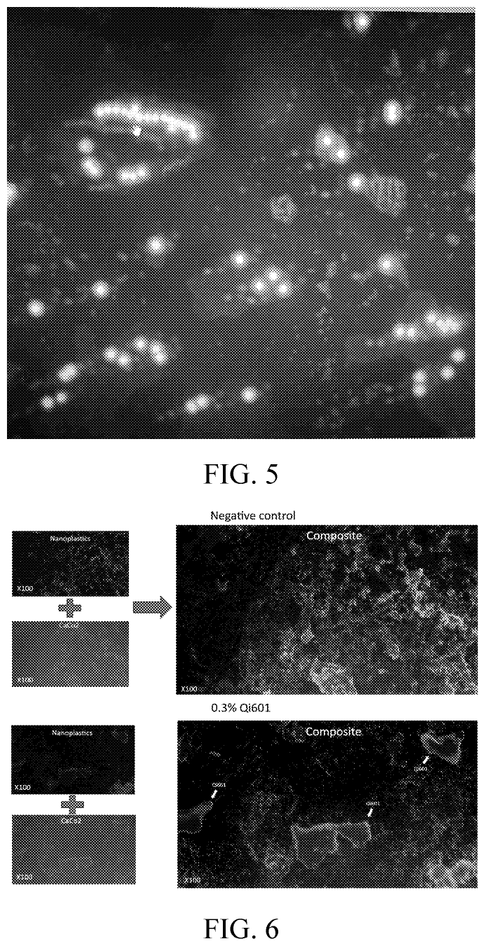

In fluorescence microscopy experiments on human colonic epithelial monolayers (the Caco 2 cell line), nano sized, green fluorescent plastic particles were applied to cells and fluorescence was observed in the cells. In untreated control conditions, fluorescent green plastic particles, ranging from 10 micron microplastics to 100 nanometer nanoplastics, formed a dense coating of plastic particles across the epithelial surface of the human colon cells, appearing as a bright green film indicative of strong plastic-human cell interaction (FIG. 6, top panels).

When Qi601 was introduced into the same experimental system, the distribution of plastic fluorescence changed dramatically (FIG. 6, right upper to right lower panels). Plastics no longer adhered to human colon cells; instead, they aggregated selectively along Qi601 biofilm structures, forming clusters distinct from the epithelial surface.

This redistribution of nanoplastic nanoparticles demonstrates the role of biofilm as a physical barrier, competitively binding nanoplastics particles to divert plastics away from human cellular contact and effectively shielding intestinal lining from direct exposure.

Quantitative analysis establishes that nanoplastic attachment to human colon cells fell by 83% with Qi601 when compared with untreated controls, evidence of a physical and functional barrier operating at the cell interface.

These results show that Qi601 functions as a protective shield inside the gut, on human colon cells, capturing plastics before they can attach to human intestinal cells.

### Example 4: Qi601 Binds Nanoplastic Suspension

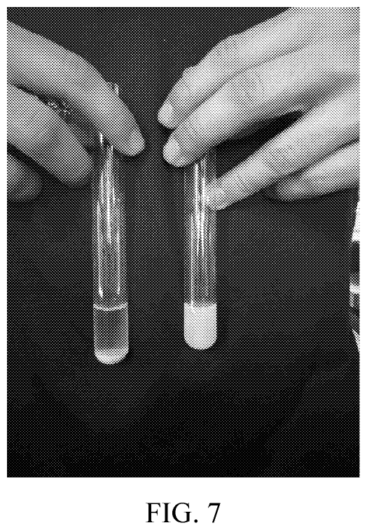

In test-tube fluorescence assays, nanoplastic suspensions glow brightly in the suspension when Qi601 is absent. (FIG. 7, right test tube) With the addition of Qi601, the bright green nanoplastic binds to Qi601 and leaves a clear liquid on top. Plastic particles are drawn down by the weight of Qi601 and incorporate into the Qi601 biofilm aggregates at the bottom of the test tube. (FIG. 7, left test tube).

This visual demonstration shows how Qi601 removes plastic contamination from its surroundings.

### Example 5: Nanoscale Imaging of Qi601 Binding Nanoplastic Particles Using Atomic Force Microscopy

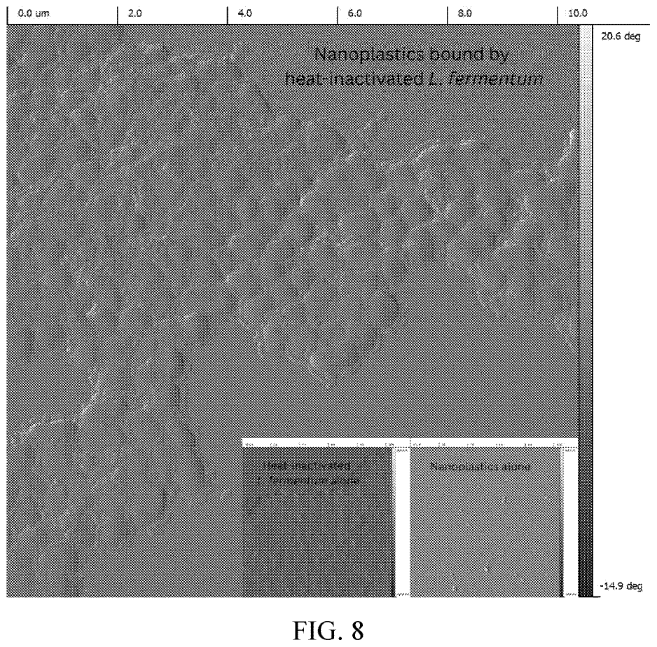

Atomic force microscopy (AFM) provides image resolution far below what the human eye can see. With nanometer-scale images, AFM revealed Qi601 bacterial biofilm aggregates as uneven cobblestone-like structures (FIG. 8, blue arrows); they form a rugged topology created by self-assembling, inactivated bacteria. (FIG. 8)

Within this cobblestone-like terrain, nanoplastic particles interdigitated on and between Qi601, (FIG. 8, red arrows) tucked into crevices and held by the biofilm matrix.

These images confirm capture of the nanoplastic particles on nanometer scale and offer a direct visualization and mechanistic confirmation of the binding capabilities of Qi601 to nano-sized plastic particles. The rough, high-surface-area architecture presents an energetically and sterically favorable binding surface and creates niches that favor nanoplastic sequestration that can outcompete plastic binding to human cells. The textured surface traps particles locking them in place and preventing them from attaching and interacting with tissue.

Because the nanoplastics do not bind to human cells, they are not absorbed into the body. Thus, the biofilm creates a first line of defense against absorbing nanoplastics across the human intestinal cellular layer.

### Example 6: In Vivo Animal Testing: Qi601 Prevents Attachment of Nanoplastics in the Gut and Promotes Excretion

Animal experiments provide additional evidence of the Qi601 anti-microplastic activity.

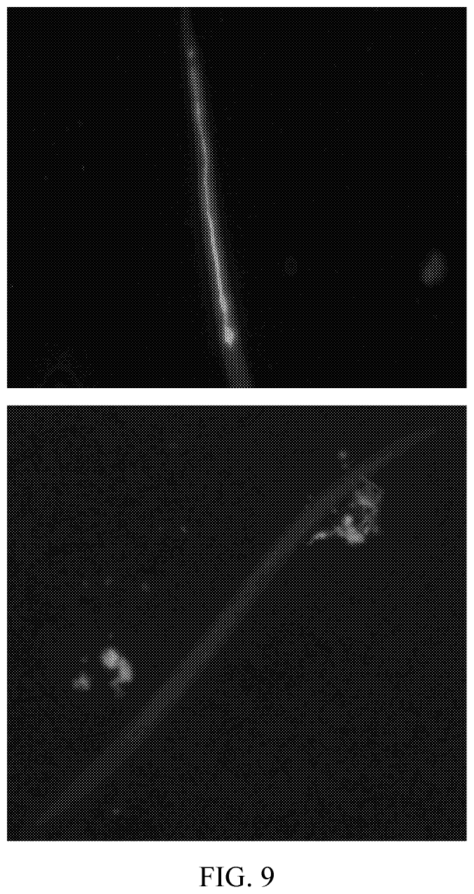

C. elegans is a transparent nematode widely used in biomedical research for testing gut barrier integrity and inflammation. C. elegans that were fed fluorescent nanoplastics had gut linings that glowed with accumulated, bright green nanoplastic particles (FIG. 9, top panel).

By contrast, the intestinal tracts of worms fed Qi601 and nanoplastics exhibited markedly reduced epithelial signal of the fluorescent nanoplastic particles (FIG. 9, bottom panel).

The nano sized fluorescence bright green nanoplastic particles present in the nematode gut in the control animals were washed out by Qi601. Excreted nanoplastics ultimately appeared in worm waste products when the worms are simultaneously fed Qi601 (FIG. 9, bottom panel, green arrow).

This visual evidence that ingested nanoplastics can be flushed out of the intestinal tract establishes that Qi601 not only binds nanoplastics in the gastric environment but also flushes the plastic particles out naturally.

This animal model demonstrates biological clearance; binding-and-elimination evidence that plastics that would normally remain in tissue and in the gut are instead bound, passed through, and expelled by attaching to Qi601.

### Example 7: Stability and Efficacy of Qi601 Binding Nanoplastics in Human Gastric Fluids

Studies were conducted using human simulated gastric fluids under both fasted and fed states. The simulated gastric fluid had an acidic pH and digestive enzymes.

Qi601 biofilm aggregates retained the activity and their structural integrity by sequestering up to 99% of introduced nanoparticles in this digestive environment.

The supernatants of the samples with and without Qi601 were measured by spectrophotometry. Once bound, plastic nanoparticles remained attached to Qi601 in the human gastric fluid environment, demonstrating a pathway in which Qi601 binds plastics before they can reach or interact with the gut lining.

This activity demonstrates the Qi601 can effectively bind nanoplastic particles from where the exposure to nanoplastics starts—in digestive fluid. Qi601 retains its binding capabilities even in stomach acid, capturing plastics so that they can safely pass out of the body.

### Example 8: Nanoplastic Internalization in Human Colon Cells without Qi601

Fluorescence microscopy of human colonic epithelial cells exposed to 100-nanometer fluorescent polystyrene (PS) nanoplastics for 24 hours reveals clear evidence of cellular uptake and intracellular accumulation.

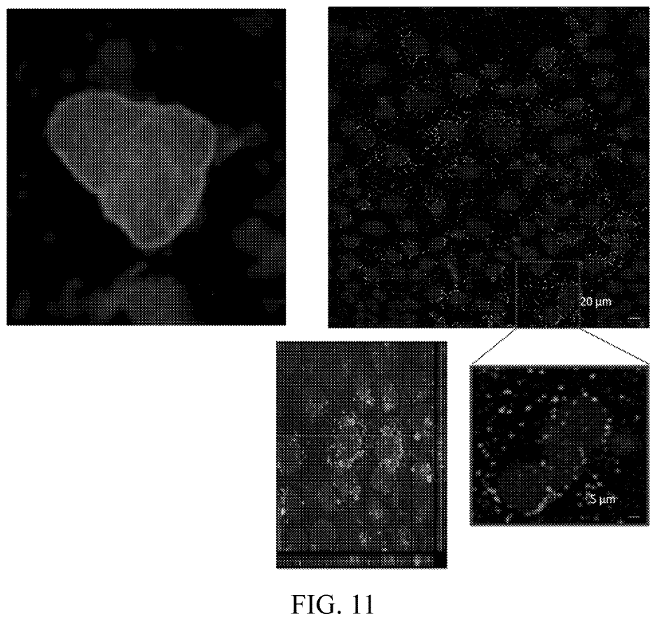

The upper left panel of FIG. 11 shows demonstrates the scale, appearance and distribution of the nanoplastics, appearing as a fine dusting of bright green fluorescent particles of the 100 nanometer nanospheres when not grouped together in endosomes. This is the typical appearance of 100 nanometer fluorescent plastics at this microscope resolution. Without Qi601 protection, punctate green fluorescent spheres are seen clustering along the nuclear and cellular boundaries within the cytoplasm, indicating that the nanoplastic particles are being actively internalized by the human colon cells (FIG. 11, top right panel). Higher-magnification (FIG. 11, bottom right panels) confirms that these particles are not merely adherent to the cell surface but likely localized within the cytoplasm and concentrated in the perinuclear region, suggesting entrapment within endosomal or lysosomal compartments.

These findings define the baseline state of nanoplastic interaction with intestinal epithelial cells, showing that in the absence of Qi601, nanoplastics can penetrate human colon cells and accumulate intracellularly. These intracellular spheres represent approximately 1000 nanoplastic particles concentrated in endosomes (FIG. 11, bottom right panel).

### Example 9: Bright Field and Fluorescent Microscopy: Qi601 Facilitates Removal of Intracellular Nanoplastics from Human Colon Cells

Bright-field and fluorescence microscopy images of human colonic epithelial cells show the ability of Qi601 to remove nanoplastics that have already entered cells.

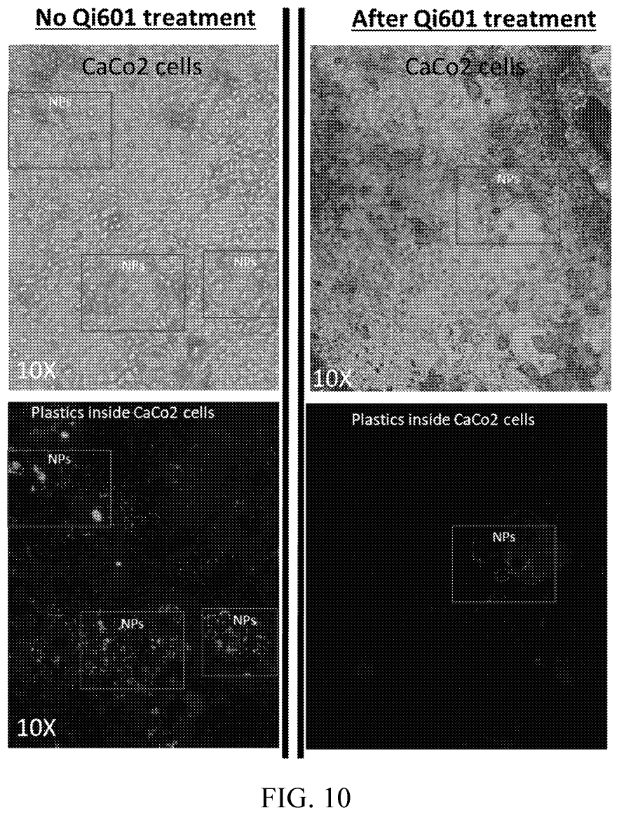

In these experiments, cells were exposed to 100 nanometer fluorescent polystyrene nanoplastic spheres (green) for 24 hours without Qi601 treatment. Cells not treated with Qi601 show numerous bright puncta, representing aggregates of approximately 1,000 to 10,000 nanoplastic particles each, visible throughout the cytoplasm, confirming intracellular accumulation (FIG. 10, left upper and lower panels).

In an identical parallel experiment, cells were first exposed to nanoplastics for 24 hours and then treated with Qi601 for an additional 4 hours. Following Qi601 treatment, intracellular fluorescence intensity markedly decreased and nanoplastic aggregates appeared diminished or absent, suggesting active removal of internalized particles by Qi601 (FIG. 10, right upper and lower panels).

These findings provide direct visual evidence that Qi601 not only prevents plastic entry into cells, but also establishes that the biofilm can also draw out, and clear, pre-existing intracellular nanoplastics.

### Example 10: Qi601 Reduces Intracellular Nanoplastics in Human Colon Cells

Fluorescence microscopy of human colonic epithelial cells demonstrates detoxifying effects of Qi601 following exposure to fluorescent 100 nanometer nanoplastics.

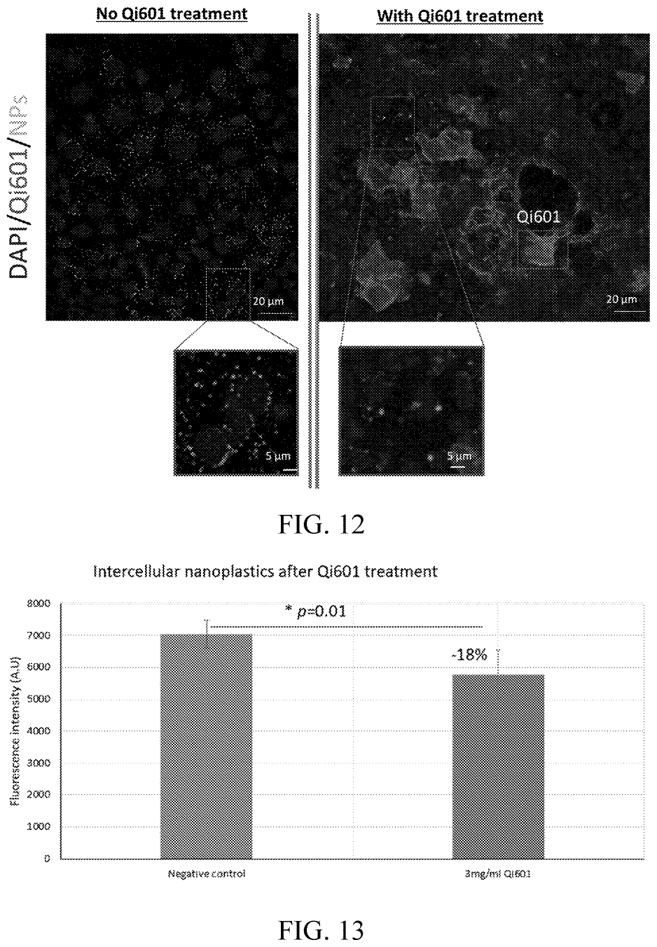

In the untreated control group (FIG. 12, left panels), cells were exposed for 24 hours to 100-nanometer fluorescent polystyrene nanoplastic spheres (green), with nuclei of the cells stained blue. The resulting images show dense green fluorescence throughout the cytoplasm, with aggregates concentrated near the nucleus, indicating perinuclear endosomal accumulation and active internalization of nanoplastic particles. The magnified view highlights the tight clustering of nanoparticles within intracellular compartments (FIG. 12, left lower panel).

In contrast, the Qi601-treated group (FIG. 12, right upper and lower panels) exposed to the same nanoplastics for 24 hours and subsequently receiving Qi601 for 4 hours exhibits a marked reduction in intracellular fluorescence. Instead, green fluorescent nanoplastic spherical particles have largely disappeared, with only remnants remaining intracellularly. (FIG. 12, right upper and lower panels). Any remaining fluorescent nanoparticles after treatment with Qi601, have been reduced to a fine dusting once they have been unbound, redistributed, and faintly clustered along extracellular Qi601 aggregates (magenta), showing that Qi601 binds and extracts plastic particles from within cells toward the extracellular environment (FIG. 12, left panel).

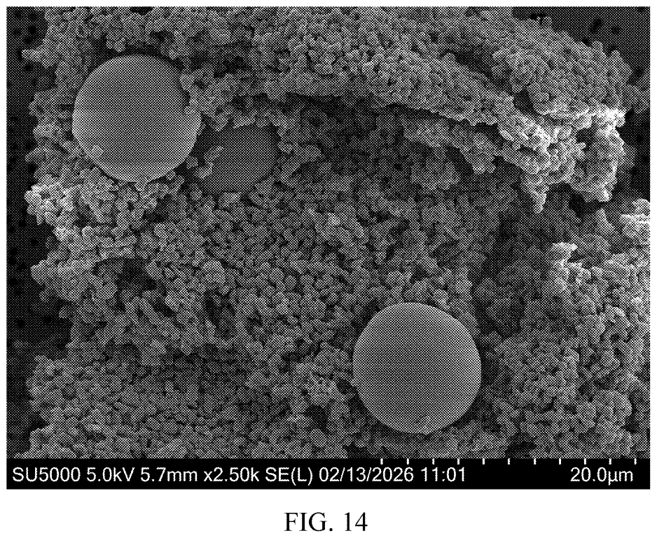

Quantitative data reinforces what the microscopy images reveal: Qi601 markedly reduces intracellular nanoplastic burden following exposure (FIG. 13). Measurements of intracellular fluorescence show a significant decline in internalized nanoplastics particles after Qi601 is introduced, mirroring the visual reduction in cytoplasmic and perinuclear nanoplastic clusters. This numerical confirmation strengthens the interpretation that Qi601's external high-affinity binding surface creates a transepithelial gradient capable of drawing particles outward, preventing their re-entry, and reducing their intracellular persistence. Together, the quantitative analyses and visual evidence converge on the same conclusion—that Qi601 actively facilitates their clearance of microplastics from inside cells.

These findings provide direct visual evidence that biofilm can function not only as a barrier that prevents nanoplastic entry into cells and tissues, but also as a detoxification agent capable of facilitating the removal of internalized plastics from human epithelial cells, effectively lowering intracellular burden even after uptake has occurred.

### Example 11: Capture of Plastic Particles by Qi601 Biofilm Matrix During Initial Oral Exposure

An interventional crossover human proof-of-concept study was conducted with five participants to evaluate the ability of Qi601 to physically associate with microplastic particles released during normal chewing.

In the control condition, participants chewed commercially-available gum for ten minutes and expectorated saliva for microscopic analysis, which demonstrated the presence of released microplastic particles. In the crossover condition, the same participants repeated the chewing procedure with Qi601 present.

Microscopic examination of the saliva samples demonstrated visible association of microplastic particles with Qi601 material. FIG. 14 is a scanning electron microscopic picture of Qi601 wrapping around 10 micron plastic spheres.

To further evaluate the stability of this association during digestion, laboratory studies were conducted using the saliva samples containing Qi601-bound nanoplastics. These samples were sequentially exposed to simulated human digestive fluids, including saliva, gastric fluid under fed and fasted conditions, upper intestinal fluid, and colonic fluid. Across this digestive sequence, approximately 98% of nanoplastic particles remained associated with Qi601 when compared with untreated controls.

Together, these findings indicate that plastic particles captured by Qi601 during initial oral exposure remain physically associated with the Qi601 biofilm matrix under conditions designed to model the human gastrointestinal environment. The results indicate that the Qi601 structure remains stable during digestion and retains bound nanoplastic particles from the point of oral exposure through gastrointestinal transit.

We claim:

1. A method for removing microplastics from a sample or an environment wherein said method comprises contacting a biofilm with a sample and/or an environment for time sufficient for the microplastics to form an aggregate with the biofilm and then removing the biofilm/microplastic aggregate from the sample and/or environment.

2. The method according to claim 1, wherein the environment is selected from a digestive tract and blood.

3. The method according to claim 1, wherein the method further comprises determining the presence and/or quantity of microplastics in the aggregate.

4. The method according to claim 1, wherein the biofilm is biofilm of Lactobacillus fermentum having accession number ATCC PTA-122195.

5. The method according to claim 1, wherein microplastics are removed from an industrial fluid system.

6. The method according to claim 1, further comprising a step of disposing of the biofilm/microplastic aggregate, wherein disposal is via landfill, incineration, or recycling.

7. The method according to claim 1, wherein said method comprises orally-administering to a subject a capsule comprising inactivated biofilm of Lactobacillus fermentum having accession number ATCC PTA-122195.

8. The method according to claim 7, further comprising detecting and/or quantifying microplastics excreted by the subject.

9. The method according to claim 1, used to remove microplastics from drinking water.

10. The method according to claim 1, wherein the biofilm has an angle greater than 100° in the static contact angle assay.

11. The method according to claim 1, wherein an intracellular substance is removed from inside a cell, wherein said method comprises contacting a cell having, or suspected of having, the intracellular substance, with a biofilm.

12. The method according to claim 11, wherein the substance is selected from microplastics, heavy metals, persistent organic pollutants, and microparticles of noncellular origin.

13. The method according to claim 11, wherein the cell is part of, or isolated from, digestive tract tissue, blood, respiratory tract tissue, or skin.

14. The method according to claim 11, wherein the substance is an exosome.

15. The method according to claim 11, which causes improved functioning of a mitochondria in said cell.

16. A capsule having encapsulated therein a composition comprising heat-inactivated Lactobacillus fermentum having accession number ATCC PTA-122195 and microcrystalline cellulose.

17. The capsule according to claim 16, wherein the composition the composition comprises 10 mg to 100 mg of said Lactobacillus fermentum.

18. A device for removing microplastics from a fluid stream wherein said device comprises a solid matrix to which a biofilm is attached, an outer housing having an inlet through which a fluid stream can be introduced into an interior of the outer housing, an outlet through which the fluid stream can exit the housing, and wherein, in said interior, is a microbial biofilm associated with the solid matrix, wherein the solid matrix is porous such that a fluid stream can pass through the matrix.

19. The device according to claim 18, wherein the matrix comprises ceramic, glass, metal and/or a polymer.

20. A method selected from:
i) a method of preventing microplastics from passing through skin wherein said method comprises applying a lotion to the skin wherein the lotion comprises a biofilm;
ii) a method for removing microplastics from a surface wherein the method comprises applying to the surface a composition comprising a biofilm and then removing the composition from the surface; and
iii) a cleansing treatment comprising contacting tissue with a composition comprising biofilm and then removing the composition thereby cleansing the tissue.
